(19) United States
(12) Patent Application Publication
(10) Pub. No.: US 2024/0240190 A1
(43) Pub. Date: Jul. 18, 2024

(54) INHIBITORS OF MITOFERRIN-2 FOR USE IN TREATING CANCER

(71) Applicants: DEUTSCHES KREBSFORSCHUNGSZENTRUM STIFTUNG DES OEFFENTLECHEN RECHTS, Heidelberg (DE); UNIVERSITAET HEIDELBERG, Heidelberg (DE)

(72) Inventors: Darjus TSCHAHARGANEH, Neckargemuend (DE); Stephen KRIEG, Heidelberg (DE)

(21) Appl. No.: 18/562,167

(22) PCT Filed: May 20, 2022
(86) PCT No.: PCT/EP2022/063693

§ 371 Date: Nov. 17, 2023

(30) Foreign Application Priority Data
May 21, 2021 (EP) 21175355.3

(51) Int. Cl. C12N 15/113; A61P 35/00; G01N 33/50

(52) U.S. Cl. CPC C12N 15/1138; A61P 35/00; G01N 33/5026; C12N 2310/14; G01N 2500/10

(57) ABSTRACT

The present invention relates to an inhibitor of mitoferrin-2 for use in treating a cancer with reduced activity of mitoferrin-1 in a subject. The present invention also relates to a method for identifying an inhibitor of mitoferrin-2 comprising (a) contacting a (i) host cell with a reduced mitoferrin-1 activity and (ii) a host cell with a non-reduced mitoferrin-1 activity with a candidate inhibitor of mitoferrin-2. (b) determining growth and/or morphology of the host cells of step (a): (c) identifying an inhibitor of mitoferrin-2 if a growth arrest and/or abnormal morphology is/are detected in step (b) in the host cell having the reduced activity of mitoferrin-1 but not in the host cell with the non-reduced activity of mitoferrin-1. The present invention further relates to a method for identifying a subject susceptible to cancer treatment by an inhibitor of mitoferrin-2, comprising (A) determining mitoferrin-1 activity in a sample of said subject, preferably a sample of cancer cells, and (B) identifying a patient susceptible to cancer treatment by an inhibitor of mitoferrin-2 based on determining step (A).

The present invention relates to an inhibitor of mitoferrin-2 for use in treating a cancer with reduced activity of mitoferrin-1 in a subject. The present invention also relates to a method for identifying an inhibitor of mitoferrin-2 comprising (a) contacting a (i) host cell with a reduced mitoferrin-1 activity and (ii) a host cell with a non-reduced mitoferrin-1 activity with a candidate inhibitor of mitoferrin-2, (b) determining growth and/or morphology of the host cells of step (a); (c) identifying an inhibitor of mitoferrin-2 if a growth arrest and/or abnormal morphology is/are detected in step (b) in the host cell having the reduced activity of mitoferrin-1 but not in the host cell with the non-reduced activity of mitoferrin-1. The present invention further relates to a method for identifying a subject susceptible to cancer treatment by an inhibitor of mitoferrin-2, comprising (A) determining mitoferrin-1 activity in a sample of said subject, preferably a sample of cancer cells, and (B) identifying a patient susceptible to cancer treatment by an inhibitor of mitoferrin-2 based on determining step (A).

Cancer cells may contain drastic changes to their genomes compared to non-cancer cells, including genes which are amplified, but also genes which are lost from the genome. Such changes are often cancer type specific or may even be subject specific. Other modifications are quite widespread, such that their identification and possible use for therapeutic approaches may benefit a large proportion of cancer patients. E.g., a deletion of the short arm of chromosome 8 (chromosome region 8p) was found to occur with frequencies of up to 70% in human cancers.

One of the genes encoded on chromosome region 8p in humans is the SLC25A37 gene, encoding mitoferrin-1, a mitochondrial iron-transporter protein. Kang et al. (2019), Autophagy 15(1): 172 described that an increased amount of mitoferrin-1 and mitoferrin-2 in cells promotes tumorigenesis. Shen et al. (2018), J Cell Biochem 119(11):9178 did not find any significant correlation between SLC25A37 gene expression and neoplasm disease stage, cancer tumor stage, histologic grade, and related tumor parameters in hepatocellular carcinoma. In contrast, Li et al. (2018), Dev Cell 46(4):441 reported that a high expression of the SLC25A37 gene is associated with poor prognosis in human pancreatic cancer; notably, this was not the case for the SLC25A28 gene encoding mitoferrin-2.

Nonetheless, there is still a need for improved methods for cancer treatment and for means and methods for identification of subjects amenable to specific treatments. This problem is solved by the embodiments characterized in the claims and described herein below.

In accordance, the present invention relates to an inhibitor of mitoferrin-2 for use in treating a cancer with reduced activity of mitoferrin-1 in a subject.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof: thus, e.g. identifying “a cell” relates to identifying at least one cell, preferably to identifying a multitude of cells. The term “multitude”, as used herein, relates to a number of two or more.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The instant description relates to gene products. The term “gene product” is known to the skilled person. Preferably, the term includes any and all products produced or producible by a host cell or by an in vitro expression system from a gene. In accordance, the term preferably includes RNA transcribed from a gene, in particular unspliced, partially spliced, and fully spliced RNA, unedited and edited RNA, as well as any polypeptide produced from one of the aforesaid RNAs, preferably mRNAs, preferably by protein biosynthesis. In accordance, the gene product preferably is an mRNA and/or a polypeptide encoded thereby. The skilled person is aware that the gene products referred to herein may be expressed in a plurality of isoforms, from different alleles, and/or may be expressed as isoforms and/or precursor forms which may be further processed in the cell, e.g. during intracellular trafficking and/or secretion. Also, the skilled person is aware that subjects from non-human species will preferably express homologues of the specific sequences indicated herein, which may preferably be identified by sequence alignment and/or search algorithms based thereon, such as the BLAST algorithm, and appropriate databases, preferably publicly available databases. Preferably, the nucleic acid or amino acid sequence of a gene product as specified is at least 50%, more preferably 75%, still more preferably 85%, even more preferably at least 95%, even more preferably at least 98%, most preferably at least 99%, identical to a specific gene product sequence as referred to herein.

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. In the context of sequence information, in particular nucleic acid sequences and/or polypeptide sequences, the term “sub-sequence” is used for sequences representing only a part of a longer sequence.

Unless specifically indicated otherwise herein, the compounds specified, in particular polynucleotides, polypeptides, or fragments thereof, e.g. inhibitors of mitoferrin-2, may be comprised in larger structures, e.g. may be covalently or non-covalently linked to accessory molecules, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C— or N— terminally to the fusion polypeptide. Said stretch of amino acids preferably allows for detection of the fusion polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences and/or linker sequences. Polynucleotides, in particular polynucleotide inhibitors of mitoferrin-2, may be comprised or produced in a cell as a sub-sequence of a longer polynucleotide, e.g. comprising further polynucleotide inhibitors of additional genes. Also, polynucleotides may be comprised in larger structures, preferably mediating cell entry, such as complexes with chemicals such as calcium chloride, diethylaminoethyl (DEAE)-comprising polymers (e.g. DEAE dextran), liposomes, virus capsids, and the like. Also, polynucleotides may be comprised in expressible constructs comprising expression control sequences, and/or in vectors. Preferably, the polynucleotide inhibitor of mitoferrin-2, more preferably the shRNA as specified herein below, is comprised in an expression construct and/or a vector, which preferably comprises, more preferably consists of, the nucleic acid sequence of SEQ ID NO:2, 4, 6, or 8.

In accordance with the above, “determining gene expression” may relate to determining any gene product as specified herein above. Expression of a gene may be determined qualitatively, semiquantitatively, or quantitatively, which terms are in principle known to the skilled person. Qualitative determination may be a binary assessment that the gene is expressed or not expressed by a cell, e.g. by determining whether the gene product is expressed above a detection level of an assay. Qualitative expression may, however, also be determining whether the gene is present in the cell; i.e., as referred to herein, a gene lacking from a cell is deemed not expressed. Semiquantitative determination may comprise assorting expression to expression categories, such as low, medium, or high expression. The term quantitative determination is understood by the skilled person to include each and every determination providing information on the amount of a gene product in a cell and all values derived from such an amount by at least one standard mathematical operation, including in particular calculation of a concentration, of a mean, a median, or an average, normalization, and similar calculations. Thus, methods of determining gene expression preferably include methods of determining an RNA as gene product, preferably mRNA, such as RNA hybridization methods; RT-PCR, preferably qRT-PCR; single-cell RNA sequencing, and the like; also, methods of determining gene expression preferably include methods of determining polypeptides, in particular immunological methods, but also reporter gene systems, which are all well known to the skilled person. Methods of determining gene expression also preferably include methods of determining a polypeptide as gene product; appropriate methods are widely known in the art and include in particular measuring activity of a polypeptide and immunological methods, such as ELISA methods and any other immunoassay methods deemed suitable by the skilled person. In view of the above, methods of determining gene expression preferably further include methods of determining the presence of a gene in a genome of a cell, e.g. by karyotyping, in situ hybridization, PCR with cellular DNA as a template, and the like. Determining gene expression preferably comprises determining the full-length gene product, more preferably comprises determining at least a fragment of at least one gene product. In case a fragment of a gene product is determined, said fragment preferably is a specific fragment, i.e. a fragment known to be only detected in case the gene is expressed. More preferably, said fragment is a unique fragment, i.e. a fragment comprising at least one nucleic acid or amino acid sequence occurring exclusively in the gene product compared to the whole genome of a cell in case the presence of a gene is determined, of the whole transcriptome of a cell in case an RNA is determined, and of the whole proteome of a cell in case a polypeptide is determined.

The term “mitoferrin-2”, as used herein, relates to the member of the solute carrier polypeptide family mediating mitochondrial iron uptake known under this designation. In humans, mitoferrin-2 is encoded by the SLC25A28 gene with chromosomal location 10q24.2 (Genbank Gene ID: 81894, HUGO Gene Nomenclature Committee (HGNC) ID: HGNC:23472). The mRNA sequence expressed from the SLC25A28 gene is available e.g. from Genbank Acc. No. NM_031212.4; the amino acid sequence of mitoferrin-2 is available e.g. from Genbank Acc. No. NP 112489.3.

The term “mitoferrin-1”, as used herein, relates to the member of the solute carrier polypeptide family mediating mitochondrial iron uptake known under this designation. In humans, mitoferrin-1 is encoded by the SLC25A37 gene with chromosomal location 8p21.2 (Genbank Gene ID: 51312, HGNC ID: HGNC:29786). The mRNA sequence expressed from the SLC25A37 gene is available e.g. from Genbank Acc. No. NM_016612.4, the amino acid sequence of mitoferrin-2 is available e.g. from Genbank Acc. No. NP_001304741.1.

The term “inhibitor of mitoferrin-2”, as used herein, includes each and every compound having the activity of inhibiting mitoferrin-2 activity; thus, the inhibitor of mitoferrin-2 preferably inhibits iron transport by mitoferrin-2 over the inner mitochondrial membrane. Preferably, said inhibition is by at least 50%, more preferably at least 75%, still more preferably at least 85%, even more preferably at least 90%, most preferably at least 95%, compared to the same cell type not treated with said inhibitor. The inhibitor of mitoferrin-2 may be a direct inhibitor or an indirect inhibitor. The term “direct inhibitor of mitoferrin-2”, as used herein, relates to a compound binding to mitoferrin-2 and thereby inhibiting the activity of mitoferrin-2.

Preferably, the direct inhibitor of mitoferrin-2 is an aptamer, an antibody, or a fragment thereof, having the activity of inhibiting mitoferrin-2. The term “indirect inhibitor of mitoferrin-2”, accordingly, includes any inhibitor of mitoferrin-2 not binding to mitoferrin-2. Thus, the indirect inhibitor of mitoferrin-2 may be a compound reducing the amount of mitoferrin-2 in a cell, e.g. by reducing expression of the SLC25A28 gene or by enhancing degradation of mitoferrin-2, by removing the SLC25A28 gene, or by reducing availability of the substrate of mitoferrin-2, i.e. intracellular iron, preferably Fe²⁺, in a cell. Preferably, the inhibitor of mitoferrin-2 is a specific mitoferrin-2 inhibitor, i.e. a compound specifically inhibiting mitoferrin, more preferably specifically inhibiting mitoferrin-2. Thus, the inhibitor of mitoferrin-2 preferably does not inhibit non-mitoferrin-2 ion transporters, preferably does not inhibit non-mitoferrin-2 mitochondrial ion transporters, more preferably does not inhibit non-mitoferrin-2 iron transporters, most preferably does not inhibit mitoferrin-1.

Preferably, the inhibitor of mitoferrin-2 comprises, preferably is, a polynucleotide, preferably having the activity of reducing expression of the gene encoding mitoferrin-2, preferably by at least 50%, more preferably at least 75%, still more preferably at least 85%, even more preferably at least 90%, most preferably at least 95%. Polynucleotides inhibiting expression of a gene of interest are provided by the skilled person according to textbook methods and are commercially available upon provision of a target sequence. Preferably, the inhibitor of mitoferrin-2 comprises a silencing polynucleotide and/or causes expression of a silencing polynucleotide in a host cell, preferably a cancer cell. The term “silencing polynucleotide”, as used herein, relates to any polynucleotide having the activity of reducing or preventing expression of a target gene, preferably SLC25A28, in a host cell. Preferably, the silencing polynucleotide is an RNA or DNA, more preferably an RNA. Preferably, the inhibitor of mitoferrin-2 comprises, preferably is, an siRNA, a shRNA, and/or an miRNA, or a polynucleotide mediating expression of at least one of the aforesaid in a host cell. Preferably, the silencing polynucleotide comprises, preferably consists of, the nucleic acid sequence 5′-TTCAGTGCTACTTCACTTGCCA-3′ (SEQ ID NO:1) or 5′-TTTAAAGGCTTTTTATTAGGAA-3′ (SEQ ID NO:3).

Preferably, the inhibitor of mitoferrin-2 is a polynucleotide inducing RNA interference. As used herein, “RNA interference”, also referred to as “RNAi” refers to sequence-specific, post-transcriptional gene silencing of a selected target gene by degradation of RNA transcribed from the target gene (target RNA). RNAi requires in the cell the presence of dsRNAs that are homologous in sequence to the target RNAs. The term “dsRNA” refers to RNA having a duplex structure comprising two complementary and anti-parallel nucleic acid strands. The RNA strands forming the dsRNA may have the same or a different number of nucleotides, whereby one of the strands of the dsRNA can be the target RNA. It is, however, also contemplated that the dsRNA is formed between two sequence stretches on the same RNA molecule, e.g. by stem-loop formation. Methods relating to the use of RNAi to silence genes in animals, including mammals, are known in the art (see, for example, Hammond et al. (2001), Nature Rev. Genet. 2, 110-119; Elbashir et al. (2001), Nature 411: 494-498). Thus, the inhibitor of mitoferrin-2 preferably is an RNAi agent. As used herein, the term “RNAi agent” refers to an shRNA, a siRNA agent, or a miRNA agent as specified below. The RNAi agent of the present invention is of sufficient length and complementarity to stably interact with the target RNA, i.e. it comprises at least 15, at least 17, at least 19, at least 21, at least 22 nucleotides complementary to the target RNA. Preferably, the inhibitor of mitoferrin-2 mediates at least one of (i) an at least partial knock-out of the gene encoding mitoferrin-2, (ii) RNA interference of mitoferrin-2 gene expression, and (iii) silencing of mitoferrin-2 gene expression.

The term “siRNA agent” as referred to herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. The term “molecule comprising a siRNA molecule” preferably includes RNA molecules from which an siRNA is processed by a cell, preferably by a mammalian cell. Thus, a molecule comprising an siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides, base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can be modulated by said expression control sequence. Preferred expression control sequences are those, which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent, e.g. cancer cell-specific expression. Preferably, the siRNA agent, more preferably the shRNA, comprises, preferably consists of, the nucleic acid sequence of SEQ ID NO: 1 or 3.

It is, however, also contemplated that the RNAi agent is a miRNA agent. A “miRNA agent” as meant herein encompasses: a) a pre-microRNA, i.e. a mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above.

Also preferably, the inhibitor of mitoferrin-2 comprises at least one CRISPR/Cas oligonucleotide, also known as guide RNA (gRNA). The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. Thus, preferably, one gRNA is used to introduce a double-strand break in the SLC25A28 gene, which is then repaired by the cell in the error-prone non-homologous end-joining (NHEJ) process, introducing a deletion/insertion into the SLC25A28 gene. More preferably, at least two gRNAs are provided, selected such that the aforesaid at least partial deletion of the SLC25A28 gene is created. Thus, preferably, the inhibitor of mitoferrin-2 comprises (i) at least one guide-RNA and (ii) a Cas nuclease or a polynucleotide causing expression of a Cas nuclease in a host cell, preferably a cancer cell. Preferably, the CRISPR/Cas oligonucleotide (gRNA) comprises, preferably consists of the nucleic acid sequence 5′-GGTGACCGCCTATTTCCGAG-3′ (SEQ ID NO:5) or 5′-TTCAGGACGGTATATCAAGT-3′ (SED ID NO:7). More preferably, the CRISPR/Cas oligonucleotide (gRNA) comprises, preferably consists of the nucleic acid sequence of SEQ ID NO: 6 or 8.

Also preferably, the inhibitor of mitoferrin-2 comprises, preferably is, a polypeptide, preferably, said polypeptide is an aptamer, an antibody, or a fragment thereof. The polypeptide may, however, also be an anticalin or a DARPin. The term “polypeptide”, as used herein, refers to a molecule comprising, preferably consisting of, a multitude of, typically at least 10, amino acids that are covalently linked to each other by peptide bonds. Molecules consisting of less than 10 amino acids covalently linked by peptide bonds may also be referred to as “peptides”. Preferably, the polypeptide comprises of from 20 to 2000, more preferably of from 50 to 1000, still more preferably of from 75 to 750, most preferably of from 100 to 500 amino acids.

Preferably, any specific peptide or polypeptide referred to herein may be comprised in a fusion polypeptide and/or a polypeptide complex.

As used herein, the term “aptamer” relates to a polypeptide binding specifically to a target molecule by virtue of its three-dimensional structure. Preferably, the aptamer comprises 8-80 amino acids, more preferably 10-50 amino acids, and most preferably 15-30 amino acids. Aptamers can e.g. be isolated from randomized peptide expression libraries in a suitable host system like baker's yeast (see, for example, Klevenz et al., Cell Mol Life Sci. 2002, 59: 1993-1998). An aptamer, preferably, is a free polypeptide; it is, however, also contemplated that an aptamer is fused to a polypeptide serving as “scaffold”, meaning that the covalent linking to said polypeptide serves to fix the three-dimensional structure of said aptamer to a specific conformation. More preferably, the aptamer is fused to a transport signal, in particular a cell-penetrating peptide.

As used herein, the term “antibody” relates to a soluble immunoglobulin from any of the classes IgA, IgD, IgE, IgG, or IgM, having the activity of directly interacting with mitoferrin-2 and inhibiting mitoferrin-2 activity as specified herein above. Antibodies against mitoferrin-2 can be prepared by well-known methods using a purified mitoferrin-2 polypeptide or a suitable fragment derived therefrom as an antigen. A fragment which is suitable as an antigen may be identified by antigenicity determining algorithms well known in the art. A fragment may also be obtained either from the mitoferrin-2 polypeptide by proteolytic digestion, may be a synthetic peptide, or may be recombinantly expressed. Suitability of an antibody thus generated as an inhibitor of mitoferrin-2 can be tested by an assay as described elsewhere herein. Preferably, the antibody of the present invention is a monoclonal antibody, a human, or humanized antibody, or primatized, chimerized or fragment thereof. More preferably, the antibody is a single chain antibody, a single-domain antibody, a nanobody, or an antibody fragment, such as Fab, scFab, and the like. Also comprised as antibodies of the present invention are a bispecific antibody, a synthetic antibody, or a chemically modified derivative of any of the aforesaid antibodies. Preferably, the antibody of the present invention shall specifically bind (i.e. does not cross react with other polypeptides or peptides) to a mitoferrin-2 polypeptide as specified above. Specific binding can be tested by various well-known techniques. Antibodies or fragments thereof can be obtained by using methods, which are described, e.g., in Harlow and Lane “Antibodies, A Laboratory Manual”, CSH Press, Cold Spring Harbor, 1988. Monoclonal antibodies can e.g. be prepared by the techniques originally described in Köhler and Milstein, Nature. 1975. 256: 495; and Galfre, Meth. Enzymol. 1981, 73: 3, which comprise the fusion of mouse myeloma cells to spleen cells derived from immunized mammals.

As used herein, the term “anticalin” relates to an artificial polypeptide derived from a lipocalin specifically binding mitoferrin-2 and inhibiting mitoferrin-2 activity. Similarly, a “Designed Ankyrin Repeat Protein” or “DARPin”, as used herein, is an artificial polypeptide, comprising several ankyrin repeat motifs, specifically binding mitoferrin-2 and inhibiting mitoferrin-2 activity.

Also preferably, the inhibitor of mitoferrin-2 comprises, preferably is, a low-molecular weight compound, preferably having a molecular weight of at most 1 kDa. Preferably, the inhibitor of mitoferrin-2 is an iron chelator. As used herein, the term “iron chelator” relates to a chemical compound forming a stable complex with iron ions, preferably Fe²⁺ and/or Fe³⁺. Preferably, the iron chelator is a compound having a log stability constant for at least one of its iron complexes of at least 3, more preferably at least 5, more preferably 10, still more preferably at least 20, even more preferably at least 25, most preferably at least 30. Preferably, the log stability constant is the log of the equilibrium constant for the formation of the Fe/iron chelator complex in aqueous solution, preferably determined under standard conditions, preferably as specified elsewhere herein; preferably, the log stability constant of an iron chelator is determined in a solution consisting of water, iron ions, and iron chelator. Iron chelators are, in principle, known in the art and include compounds comprising at least one of a 2-pyridone structure, a hydroxamate structure, a (thio)semicarbazone structure, a bis(2-hydroxyphenyl)-1H-1,2,4-triazol structure, an alpha-hydroxyketone structure, an arylhydrazone structure, and a catechol structure.

The term “pharmaceutically compatible”, as used herein, relates to a chemical compound which is pharmaceutically acceptable in the sense of being not deleterious to the recipient thereof and, preferably, being compatible with optional other ingredients of a formulation thereof. Preferably, a pharmaceutically compatible compound is a compound causing at most moderate adverse drug reactions, preferably causing at most mild adverse drug reactions. As used herein, the term “mild” adverse reactions relates to adverse reactions not requiring medical intervention, such as skin rashes, headaches, digestive disturbances, fatigue, and the like; “moderate” adverse reactions are adverse reactions requiring medical intervention, but not being potentially life threatening.

In accordance, a “pharmaceutically compatible iron chelator” is an iron chelator as specified herein above which is pharmaceutically compatible as specified above. Thus, preferably, the pharmaceutically compatible iron chelator is an iron chelator comprising a chemical compound in clinical use, preferably approved for clinical use by at least one of the Food and Drug Administration (FDA), the European Medicines Agency (EMEA), and the Bundesinstitut für Arzneimittel und Medizinprodukte (BfArM). More preferably, the pharmaceutically compatible iron chelator is an iron chelator comprising a chemical compound in clinical use as an iron chelator, preferably approved for clinical use by at least one of the aforesaid institutions. Thus, preferably, the iron chelator is ciclopirox (2(1H)-Pyridinone, 6-cyclohexyl-1-hydroxy-4-methylpyridin-2(1H)-one; CAS-No: 29342-05-0), Deferoxamine (DFO, CAS No. 70-51-9) or hydroxycarbamide (CAS No. 127-07-1), nitrofural (CAS No. 59-87-0), 3-aminopyridine-2-carboxaldehyde Thiosemicarbazone (Triapine, CAS No. 236392-56-6) or 5-Hydroxypyridine-2-carboxaldehyde Thiosemicarbazone (HPCT, CAS No. 19494-89-4), Deferasirox (CAS No. 201530-41-8), iv) Deferiprone (Cas No. 30652-11-0), or N,N′N″-tris(2-pyridylmethyl)-cis,cis-1,3,5-triaminocyclohexane (Tachpyr).

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating cancer is reducing tumor burden in a subject. As will be understood by the skilled person, effectiveness of treatment of e.g. cancer is dependent on a variety of factors including, e.g. cancer stage and cancer type. Preferably, treating has the effect of causing a tumor to stop growing, more preferably to cause regression of a tumor, more preferably of causing a tumor to resolve.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a solid cancer, a metastasis, or a relapse thereof. Cancer may be induced by an infectious agent, preferably a virus, more preferably an oncogenic virus, more preferably Epstein-Barr virus, a hepatitis virus, Human T-lymphotropic virus 1, a papillomavirus, or Human herpesvirus 8. Cancer may, however, also be induced by chemical compounds, e.g. a carcinogen, or endogenously, e.g. caused by spontaneous mutation.

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is a solid cancer, a metastasis, or a relapse thereof. More preferably, said cancer is liver cancer, preferably hepatocellular carcinoma; lung cancer, preferably lung adenocarcinoma; pancreas cancer, preferably pancreas adenocarcinoma; or colon cancer, preferably colon adenocarcinoma. Also preferably, the cancer is another adenocarcinoma, e.g. breast adenocarcinoma, esophageal adenocarcinoma, prostate adenocarcinoma, cervical adenocarcinoma, or stomach adenocarcinoma.

The term “reduced activity” of a compound, preferably mitoferrin-1, is understood by the skilled person. Preferably, the activity is reduced by at least 50%, more preferably at least 75%, still more preferably at least 85%, even more preferably at least 90%, most preferably at least 95%, compared to control cells, preferably non-cancer cells, more preferably SNU387 cells. Reduced activity of mitoferrin-1 may be measured by measuring the iron transport activity of mitoferrin-1 in a cell. More preferably, activity of mitoferrin-1 in a cell is determined by determining the amount of mitoferrin-1 polypeptide and/or of mitoferrin-1 encoding mRNA in said cell; the activity of mitoferrin-1 may, however, also be determined by determining the presence of a mitoferrin-1 encoding gene in a cell. Thus, as used herein, a cell not comprising a SLC25A37 gene or not expressing a SLC25A37 gene is a cell with a reduced mitoferrin-1 activity. Also, a cell comprising only one copy of the SLC25A37 gene, i.e. preferably comprising a heterozygous deletion of the SLC25A37 gene, is deemed to have a reduced mitorferrin-1 activity, preferably redued by at least 50%. Thus, preferably, a cancer with reduced activity of mitoferrin-1 is a cancer with reduced expression of the gene encoding mitoferrin-1. Preferably, the subject is a human and the cancer is a cancer comprising a deletion of chromosomal region 8p21.2, more preferably of chromosome 8p.

The term “subject”, as used herein, relates to an animal, preferably a vertebrate, more preferably a mammal, preferably to a livestock, like a cattle, a horse, a pig, a sheep, or a goat, to a companion animal, such as a cat or a dog, or to a laboratory animal, like a rat, mouse, or guinea pig. Preferably, the mammal is a primate, more preferably a monkey, most preferably a human. Preferably, the subject is suffering from cancer, preferably a solid cancer, more preferably is suffering from liver cancer, lung cancer, pancreas cancer, or colon cancer, most preferably comprising a chromosome 8p deletion.

Advantageously, it was found in the work underlying the present invention that iron uptake in mitochondria is dependent on the activity of mitoferrin-1 and mitoferrin-2 and that upon loss of mitoferrin-1 activity, e.g. by a chromosome 8p deletion, cells, in particular cancer cells, become highly sensitive to mitoferrin-2 inhibition. Thus, cancers with a chromosome 8p deletion are treatable with inhibitors of mitoferrin-2; also, inhibition of mitoferrin-1 in cells makes it possible to screen for inhibitors of mitoferrin-2.

The definitions made above apply mutatis mutandis to the following. Additional definitions and explanations made further below also apply for all embodiments described in this specification mutatis mutandis.

The present invention further relates to a method for identifying an inhibitor of mitoferrin-2 comprising
(a) contacting a (i) host cell with a reduced mitoferrin-1 activity and (ii) a host cell with a non-reduced mitoferrin-1 activity with a candidate inhibitor of mitoferrin-2,
(b) determining growth and/or morphology of the host cells of step (a);
(c) identifying an inhibitor of mitoferrin-2 if a growth arrest and/or abnormal morphology is/are detected in step (b) in the host cell having the reduced activity of mitoferrin-1 but not in the host cell with the non-reduced activity of mitoferrin-1.

The method for identifying an inhibitor of mitoferrin-2 of the present invention, preferably, is an in vitro method. Moreover, it may comprise steps in addition to those explicitly mentioned above. For example, further steps may relate, e.g., to providing host cells for step (a). Moreover, one or more of said steps may be assisted or performed by automated equipment. As will be understood by the skilled person, the method for identifying an inhibitor of mitoferrin-2 as specified is particularly suited for identifying a specific mitoferrin-2 inhibitor.

The term “host cell”, as used herein, relates to a vertebrate cell, preferably a mammalian cell, even more preferably a human cell, comprising mitoferrin-2 activity. Preferably, the host cell is a cultured cell, preferably a cell line. The term “host cell with a reduced mitoferrin-1 activity” is, in view of the description herein, understood by the skilled person. Thus, the host cell with a reduced mitoferrin-1 activity preferably is a host cell contacted with a specific mitoferrin-1 inhibitor, e.g. a silencing polynucleotide as specified herein above, a cell not expressing a gene encoding mitoferrin-1, and/or a cell lacking a gene encoding mitoferrin-1, e.g. a human cell having a chromosome 8p deletion. In accordance, a “host cell with a non-reduced mitoferrin-1 activity” is a cell with a mitoferrin-1 activity in the range naturally occurring in body cells of an apparently healthy subject. Thus, the host cell with a non-reduced mitoferrin-1 activity preferably is a host cell not contacted with a specific mitoferrin-1 inhibitor and expressing a gene encoding mitoferrin-1. Preferably, the host cell with a reduced mitoferrin-1 activity and the host cell with a non-reduced mitoferrin-1 activity are similar, preferably essentially identical except for mitoferrin-1 expression. Thus, the host cell with a reduced mitoferrin-1 activity may be a tumor cell comprising a chromosome 8p deletion and the host cell with a non-reduced mitoferrin-1 activity may be a cell of a tissue surrounding said tumor and not comprising a chromosome 8p deletion. Preferably, the host cell with a reduced mitoferrin-1 activity is a tumor cell comprising a chromosome 8p deletion and the host cell with a non-reduced mitoferrin-1 activity is a human cell comprising a chromosome 8p deletion expressing a gene encoding mitoferrin-1. The host cell with a reduced mitoferrin-1 activity may, however, also be a host cell, e.g. a cell line, contacted with a specific inhibitor of mitoferrin-1, e.g. a silencing polynucleotide, while the host cell with a non-reduced mitoferrin-1 activity may be said host cell not contacted with a specific inhibitor of mitoferrin-1.

The term “candidate inhibitor of mitoferrin-2” may relate to any chemical compound for which the skilled person may assume that it may be an inhibitor of mitoferrin-2. Thus, the candidate inhibitor of mitoferrin-2 preferably is a biological macromolecule, e.g. a polynucleotide, polypeptide, preferably as specified herein above, or a polysaccharide, or the like. More preferably, the candidate inhibitor of mitoferrin-2 is a low-molecular weight compound as specified herein above.

The term “determining growth” of a host cell is understood by the skilled person. Preferably, the term relates to determining cell proliferation, i.e. determining whether the number of cells in a culture increases, remains constant, or decreases. Also, the term “determining morphology” of a host cell is understood by the skilled person. Preferably, the term includes determining cell size, cell shape, number and/or shape or organelles, number and/or shape of cell extensions, and the like.

An inhibitor of mitoferrin-2 is identified based on the results of determining growth and/or morphology of host cells. Thus, preferably, the results of said determination for host cells with a reduced mitoferrin-1 activity are compared to those of host cells with a non-reduced mitoferrin-1 activity as a reference. Thus, if a growth arrest, cells lysis, and/or abnormal morphology is/are detected in step (b) in the host cell having the reduced activity of mitoferrin-1 but not in the host cell with the non-reduced activity of mitoferrin-1, the candidate compound is identified to be an inhibitor of mitoferrin-2.

Further, the present invention relates to a method for identifying a subject susceptible to cancer treatment by an inhibitor of mitoferrin-2, comprising
(A) determining mitoferrin-1 activity in a sample of said subject, and
(B) identifying a patient susceptible to cancer treatment by an inhibitor of mitoferrin-2 based on determining step (A).

The method for identifying a subject of the present invention, preferably, is an in vitro method. Moreover, it may comprise steps in addition to those explicitly mentioned above. For example, further steps may relate, e.g., to providing a sample of cancer cells for step (A), and/or to providing cancer treatment to said subject, preferably comprising administration of an inhibitor of mitoferrin-2 as specified herein above. Moreover, one or more of said steps may be assisted or performed by automated equipment.

Methods for determining expression of mitoferrin-1 activity have been described herein above in the context of determining reduced mitoferrin-1 activity. In accordance, determining mitoferrin-1 activity preferably comprises determining expression of the gene encoding mitoferrin-1, more preferably comprises determining the presence of a mitoferrin-1 encoding gene in a cells.

The term “sample” refers to a sample of separated cells or to a sample from a tissue or an organ, preferably from a tumor. Thus, the sample preferably comprises or is assumed to comprise cancer cells, preferably tumor cells. Thus, the sample preferably is a tumor sample, e.g. a biopsy. As is known to the skilled person, tissue or organ samples may be obtained from any tissue or organ by, e.g., biopsy, surgery, or any other method deemed appropriate by the skilled person. Separated cells may be obtained from the body fluids, such as lymph, blood, plasma, serum, liquor and other, or from the tissues or organs by separating techniques such as centrifugation or cell sorting. Preferably, the sample is a tissue or body fluid sample which comprises cells. Preferably the sample is a sample of a body fluid, preferably a blood sample.

The body fluid sample can be obtained from the subject by routine techniques which are well known to the person skilled in the art, e.g., venous or arterial puncture, lavage, or any other method deemed appropriate by the skilled person.

A patient susceptible to cancer treatment by an inhibitor of mitoferrin-2 is determined based on the result of determining mitoferrin-1 activity. In accordance, in case it is determined that mitoferrin-1 activity, gene expression, and/or gene dosage is reduced by at least 50%, preferably at least 75%, more preferably at least 85%, even more preferably at least 90%, most preferably at least 95%, compared to a reference, the subject is identified as being susceptible to cancer treatment by an inhibitor of mitoferrin-2. The reference to which mitoferrin-1 activity is compared to preferably is a non-cancer cell, e.g. of a cancer-adjacent non-caner tissue. The reference may, however, also be derived from cells known not to have a reduced mitoferrin-1 activity, e.g. a cell line, or may be an average of expression over a set of subjects or cell lines. Also preferably, the reference is a sample of cells known to be susceptible to treatment by an inhibitor of mitoferrin-2; in such case, a patient susceptible to cancer treatment by an inhibitor of mitoferrin-2 is preferably identified if the expression determined in step (A) is essentially identical to or lower than the reference. Also preferably, the reference is a sample of cells known not to be susceptible to treatment by an inhibitor of mitoferrin-2; in such case, a patient susceptible to cancer treatment by an inhibitor of mitoferrin-2 is preferably identified if the expression determined in step (A) is lower than the reference. Preferably, a subject is identified as susceptible to cancer treatment by an inhibitor of mitoferrin-2 in case the at least one, preferably both, alleles of the gene encoding mitoferrin-1, preferably the SLC25A37 gene in case of a human cell, is found to be lacking from a cell, e.g. by in situ hybridization and/or karyotyping. Thus, the method may comprise a step of performing in situ hybridization and or karyotyping of a sample of cancer cells as step (A), and determining whether said sample comprises cancer cells having a chromosome 8p deletion. Step (a) may, however, also comprise immunohistochemical staining cancer cells for mitoferrin-1.

The present invention also relates to a kit comprising a means for determining mitoferrin-1 gene expression and an inhibitor of mitoferrin-2.

The term “kit”, as used herein, refers to a collection of the aforementioned compounds, means or reagents, which may or may not be packaged together. Preferably, the inhibitor of mitoferrin-2 is comprised in a composition, preferably as a medicament, in the kit. The housing may be any kind of container and/or packaging deemed appropriate by the skilled person. The components of the kit may be comprised by separate vials (i.e. as a kit of separate parts) or provided in a single vial. Preferably, the housing is adapted such that the components of the kit may be transported together as a unit. Moreover, it is to be understood that the kit, preferably, is to be used for practicing the methods referred to elsewhere herein. It is, preferably, envisaged that all components are provided in a ready-to-use manner for practicing the methods referred to above. Further, the kit, preferably, contains instructions for carrying out said methods. The instructions can be provided by a user's manual in paper or electronic form. In addition, the manual may comprise instructions for administration and/or dosage instructions for carrying out the aforementioned methods using the kit of the present invention. Preferably, the kit comprises a diluent and/or a means of administration. Appropriate diluents are known to the skilled person; means of administration are all means suitable for administering the inhibitor of mitoferrin-2 to a subject. The means of administration may include a delivery unit for the administration of the compound and a storage unit for storing said compound until administration. However, it is also contemplated that the means of the current invention may appear as separate devices in such an embodiment and are, preferably, packaged together in said kit. Preferred means for administration are those which can be applied without the particular knowledge of a specialized technician. In a preferred embodiment, the means for administration is a syringe, more preferably with a needle, comprising the compound or composition of the invention. In another preferred embodiment, the means for administration is an intravenous infusion (IV) equipment comprising the compound or composition.

The present invention further relates to a method for treating a subject suffering from cancer comprising administering an inhibitor of mitoferrin-2 to said subject.

The present invention also relates to a use of an inhibitor of mitoferrin-2 for the manufacture of a medicament for treating cancer.

The terms “pharmaceutical composition” and “medicament”, as used herein, relate to a composition comprising the compound or compounds as specified herein in a pharmaceutically acceptable form and, preferably, a pharmaceutically acceptable carrier. The compounds can be formulated as pharmaceutically acceptable salts. Acceptable salts comprise acetate, methylester, HCl, sulfate, chloride and the like. The pharmaceutical compositions are, preferably, administered topically or systemically. Suitable routes of administration conventionally used for drug administration are oral, intravenous, or parenteral administration as well as inhalation. Preferably, the pharmaceutical composition of the present invention is administered via a parenteral route, preferably subcutaneously, intramuscularly, or intraperitoneally. However, polynucleotide compounds may also be administered in a gene therapy approach by using viral vectors, viruses or liposomes, and may also be administered topically, e.g. as an ointment or intratumorally. Moreover, the compounds can be administered in combination with other drugs either in a common pharmaceutical composition or as separated pharmaceutical compositions wherein said separated pharmaceutical compositions may be provided in form of a kit of parts. In particular, co-administration of a chemotherapeutic agent is envisaged.

The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The diluent(s) is/are preferably selected so as not to affect the biological activity of the inhibitor of mitoferrin-2 and potential further pharmaceutically active ingredients. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats a condition referred to herein. Therapeutic efficacy and toxicity of compounds can be determined by standard pharmaceutical procedures in cell culture or in experimental animals, e.g., by determining the ED50 (the dose therapeutically effective in 50% of the population) and/or the LD50 (the dose lethal to 50% of the population). The dose ratio between therapeutic and toxic effects is the therapeutic index, and it can be expressed as the ratio, LD50/ED50. The dosage regimen will be determined by the attending physician, preferably taking into account relevant clinical factors and, preferably, in accordance with any one of the methods described elsewhere herein. As is well known in the medical arts, a dosage for any one patient may depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 μg to 10000 μg; however, doses below or above this exemplary range are envisioned, especially considering the aforementioned factors. Generally, the regimen comprises administration of 1 μg to 10 mg of a compound, however, depending on the subject and the mode of administration, the quantity of substance administration may vary over a wide range to provide from about 0.01 mg per kg body mass to about 1 mg per kg body mass, preferably. The pharmaceutical compositions and formulations referred to herein are administered at least once in order to treat a disease or condition recited in this specification. However, the said pharmaceutical compositions may be administered more than one time, for example, preferably from one to four times, more preferably two or three times. Depending on the specific type of inhibitor, the pharmaceutical composition may also be administered periodically, e.g. once a week, daily, or two times a day.

Specific pharmaceutical compositions are prepared in a manner well known in the pharmaceutical art and comprise at least an inhibitor of mitoferrin-2 as an active compound in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescriber or user instructions in order to anticipate dose adjustments depending on the considered recipient.

In view of the above, the following embodiments are particularly envisaged:

Embodiment 1: An inhibitor of mitoferrin-2 for use in treating a cancer with reduced activity of mitoferrin-1 in a subject.

Embodiment 2: The inhibitor of mitoferrin-2 for use of embodiment 1, wherein said subject is a human.

Embodiment 3: The inhibitor of mitoferrin-2 for use of embodiment 1 or 2, wherein said cancer with reduced activity of mitoferrin-1 is a cancer with reduced expression of the gene encoding mitoferrin-1.

Embodiment 4: The inhibitor of mitoferrin-2 for use of any one of embodiments 1 to 3, wherein said cancer with reduced activity of mitoferrin-1 is a cancer comprising a deletion of chromosome 8p.

Embodiment 5: The inhibitor of mitoferrin-2 for use of any one of embodiments 1 to 4, wherein said cancer is a solid cancer, preferably is liver cancer, lung cancer, pancreas cancer, or colon cancer.

Embodiment 6: The inhibitor of mitoferrin-2 for use of any one of embodiments 1 to 5, wherein said inhibitor of mitoferrin-2 comprises a polynucleotide.

Embodiment 7: The inhibitor of mitoferrin-2 for use of any one of embodiments 1 to 6, wherein said polynucleotide has the activity of reducing expression of the gene encoding mitoferrin-2.

Embodiment 8: The inhibitor of mitoferrin-2 for use of any one of embodiments 1 to 7, wherein said inhibitor of mitoferrin-2 comprises a silencing polynucleotide and/or mediates expression of a silencing polynucleotide in a host cell, preferably a cancer cell, preferably wherein said inhibitor of mitoferrin-2 comprises the nucleic acid sequence of SEQ ID NO: 1 or 3.

Embodiment 9: The inhibitor of mitoferrin-2 for use of any one of embodiments 1 to 8, wherein said inhibitor of mitoferrin-2 mediates at least one of (i) an at least partial knock-out of the gene encoding mitoferrin-2, (ii) RNA interference of mitoferrin-2 gene expression, and (iii) silencing of mitoferrin-2 gene expression.

Embodiment 10: The inhibitor of mitoferrin-2 for use of any one of embodiments 1 to 9, wherein said inhibitor of mitoferrin-2 comprises an siRNA, a shRNA, and/or an miRNA.

Embodiment 11: The inhibitor of mitoferrin-2 for use of any one of embodiments 1 to 7, wherein said inhibitor of mitoferrin-2 comprises (i) at least one guide-RNA and (ii) a Cas nuclease or a polynucleotide causing expression of a Cas nuclease in a host cell, preferably a cancer cell, preferably wherein said gRNA comprises the nucleic acid sequence of SEQ ID NO:5 or 7.

Embodiment 12: The inhibitor of mitoferrin-2 for use of any one of embodiments 1 to 11, wherein said inhibitor comprises a polypeptide.

Embodiment 13: The inhibitor of mitoferrin-2 for use of any one of embodiments 1 to wherein said inhibitor comprises an aptamer, an antibody, or a fragment thereof.

Embodiment 14: The inhibitor of mitoferrin-2 for use of any one of embodiments 1 to 13, wherein said inhibitor of mitoferrin-2 comprises a low-molecular weight compound, preferably having a molecular weight of at most 1 kDa.

Embodiment 15: The inhibitor of mitoferrin-2 for use of any one of embodiments 1 to 14, wherein said inhibitor of mitoferrin-2 is an iron chelator.

Embodiment 16: The inhibitor of mitoferrin-2 for use of any one of embodiments 1 to wherein said inhibitor of mitoferrin-2 is a specific inhibitor of mitoferrin-2.

Embodiment 17: A method for identifying an inhibitor of mitoferrin-2 comprising
(a) contacting a (i) host cell with a reduced mitoferrin-1 activity and (ii) a host cell with a non-reduced mitoferrin-1 activity with a candidate inhibitor of mitoferrin-2,
(b) determining growth and/or morphology of the host cells of step (a);
(c) identifying an inhibitor of mitoferrin-2 if a growth arrest and/or abnormal morphology is/are detected in step (b) in the host cell having the reduced activity of mitoferrin-1 but not in the host cell with the non-reduced activity of mitoferrin-1.

Embodiment 18: The method of embodiment 17, wherein said cell is a vertebrate cell, preferably a mammalian cell, more preferably a human cell.

Embodiment 19: The method of embodiment 17 or 18, wherein said cell with a reduced mitoferrin-1 activity is a human cell comprising a chromosome 8p deletion.

Embodiment 20: The method of any one of embodiments 17 to 19, wherein said cell with a non-reduced mitoferrin-1 activity is a human cell comprising a chromosome 8p deletion expressing a gene encoding mitoferrin-1.

Embodiment 21: A method for identifying a subject susceptible to cancer treatment by an inhibitor of mitoferrin-2, comprising
(A) determining mitoferrin-1 activity in a sample of said subject, and
(B) identifying a patient susceptible to cancer treatment by an inhibitor of mitoferrin-2 based on determining step (A).

Embodiment 22: The method of embodiment 21, wherein said sample is a sample of cancer cells.

Embodiment 23: The method of embodiment 21 or 22, wherein said subject is a human.

Embodiment 24: The method of any one of embodiments 21 to 23, wherein mitoferrin-1 expression is determined by determining whether said sample comprises cancer cells having a chromosome 8p deletion.

Embodiment 25: The method of any one of embodiments 21 to 24, wherein mitoferrin-1 gene expression is compared to a reference.

Embodiment 26: The method of any one of embodiments 21 to 25, wherein said reference is a sample of cells known to be susceptible to treatment by an inhibitor of mitoferrin-2, and wherein a patient susceptible to cancer treatment by an inhibitor of mitoferrin-2 is identified if the expression determined in step (A) is essentially identical to or lower than the reference.

Embodiment 27: The method of any one of embodiments 21 to 26, wherein said reference is a sample of cells known not to be susceptible to treatment by an inhibitor of mitoferrin-2, and wherein a patient susceptible to cancer treatment by an inhibitor of mitoferrin-2 is identified if the expression determined in step (A) is lower than the reference.

Embodiment 28: A kit comprising a means for determining mitoferrin-1 gene expression and an inhibitor of mitoferrin-2.

Embodiment 29: A method for treating a subject suffering from cancer comprising administering an inhibitor of mitoferrin-2 to said subject.

Embodiment 30: Use of an inhibitor of mitoferrin-2 for the manufacture of a medicament for treating cancer.

Embodiment 31: The method of embodiment 29 and/or the use of embodiment 30, having a feature of any one of embodiments 2 to 16.

All references cited in this specification are herewith incorporated by reference with respect to their entire disclosure content and the disclosure content specifically mentioned in this specification.

### Figure Legends

FIG. 1: MFRN1 and MFRN2 expression across cancer cell lines. (A) Immunoblot showing MFRN1 protein level in common human liver cancer cell lines. Vinculin was used as loading control. (B) MFRN1 and MFRN1 mRNA expression level in common liver cancer cell lines and (C) primary mouse liver cancer cell lines.

FIG. 2: MFRN1-2KO leads to distinct growth arrest and morphological change. (A) Westernblot for MFRNI in SNU387 cells transfected with a CRISPR/Cas9 expression construct containing guides targeting MFRNI (MFRNIKO), MFRN2 (MFRN2KO) or both MFRNI and MFRN2 (MFRN1-2KO). (B) Brightfield images of SNU387 cells 8 days after CRISPR/Cas9 mediated KO of MFRNI (MFRNIKO), MFRN2 (MFRN2KO) or both (MFRN1-2KO). EV=empty vector, was used as control.

FIG. 3: Cellular MFRNI status effects response to MFRN2-KO. (A) Competition assay of HUH6 cells (MFRN1-low expresser) or (B) SNU387 cells (MFRN1-high expresser) showing competitive cell growth of MFRNIKO cells with either MFRNIKO (MFRN1+MFRNI/EV), MFRN2KO (MFRN1+MFRN2/EV) or MFRN1-2KO (MFRN1+MFRN1/MFRN2). Cells were transfected with a lentiviral CRISPR/Cas9 expression contruct containing a sgRNA targeting MFRNI or no sgRNA (EV). These cells were again transfected with a lentiviral GFP expressing construct containing a second sgRNA targeting MFRN2 or no sgRNA (EV). These genetically modified cells were mixed with MFRNIKO cells and GFP positivity measured with the the Guava® easyCyte. Lower panel: Colony formation assay of HUH6 cells (left) and SNU387 cells (right) containing indicated genetic modifications.

FIG. 4: Effects of shRNA mediated knockdown of MFRN2 on HUH6 cells. (A) Colony formation assays showing clonogenic cell growth of HUH6 cells transfected with a Tet-incucible lentiviral construct expressing an shRNA targeting Renilla (shRenilla) or MFRN2 (MFRN2.1, MFRN2.2) and GFP Doxycycline (DOX) dependently. (B) Competition assay of HUH6 cells showing competitive cell growth of HUH6 cells expressing either shRenilla or an shRNA targeting MFRN2 (MFRN2.1,MFRN2.2) mix with nonmodified HUH6 cells in DOX containing medium. (C) Westernblot for MFRN2 in HUH6 cells transfected with the respective inducible shRNA constructs (shRenilla, MFRN2.1, MFRN2.2) grown in DOX containing medium or under DOX withdrawal.

FIG. 5: Recapitulating loss of MFRNI and MFRN2 in vivo using a mouse xenograft model. (A) Schematic illustration of the workflow from cell preparation to sc, injection of immunodeficient mice. (B) Upper panel: Tumor volume change over time for each individual tumor xenograft harboring the indicated alterations. Lower panel: Stereomicroscopic imaging with macroscopic pictures in Brightfield (BF) exposure and GFP-channel. Immunohistochemical staining of GFP of same samples with different magnifications. Scalebar: 500 μm and 100 μm.

FIG. 6: Recapitulating loss of MFRNI and MFRN2 in vivo using a mouse xenograft model. Left panel: Tumor volume change over time for each individual tumor xenograft harboring the indicated alterations. Right panel: Stereomicroscopic imaging with macroscopic pictures of exemplary tumors in Brightfield (BF) exposure and GFP-channel. Immunohistochemistry staining of GFP (IHC a-GFP) of same samples with different magnifications. Scalebar: 500 μm and 100 μm.

FIG. 7: Recapitulating loss of MFRNI and MFRN2 in vivo using a mouse xenograft model. Left panels: Tumor volume change over time for each individual tumor xenograft harboring the indicated alterations. Right panel: Stereomicroscopic imaging with macroscopic pictures of exemplary tumors in Brightfield (BF) exposure and GFP-channel. Immunohistochemistry staining of GFP (IHC a-GFP) of same samples with different magnifications. Scalebar: 500 μm and 100 μm; OE: overexpression.

FIG. 8: MFRN1 expression levels in normal liver tissue samples and in liver cancer samples. Representative images of patient samples individually scored (MFRN1-Score) according to their staining intensities as indicated in Example 10; HCC: hepatocellular carcinoma.

The following Examples shall merely illustrate the invention. They shall not be construed, whatsoever, to limit the scope of the invention.

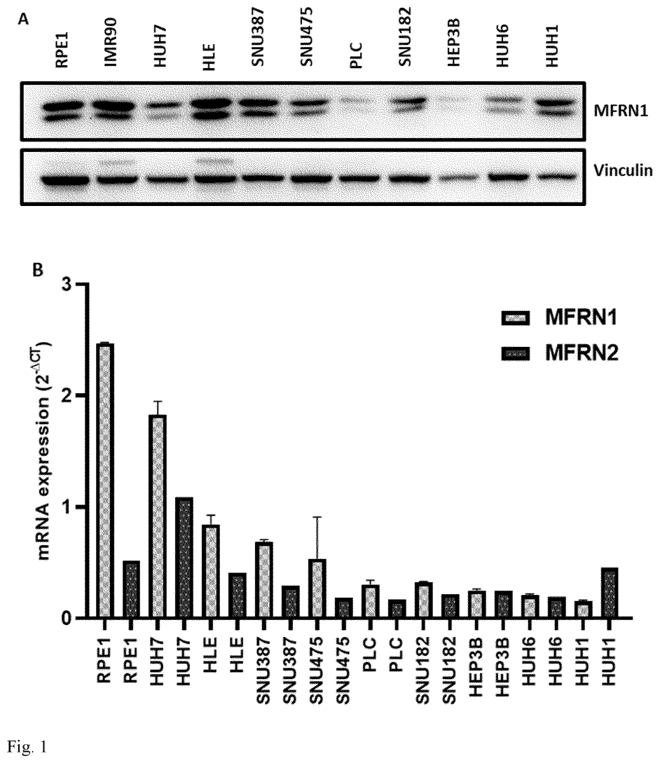

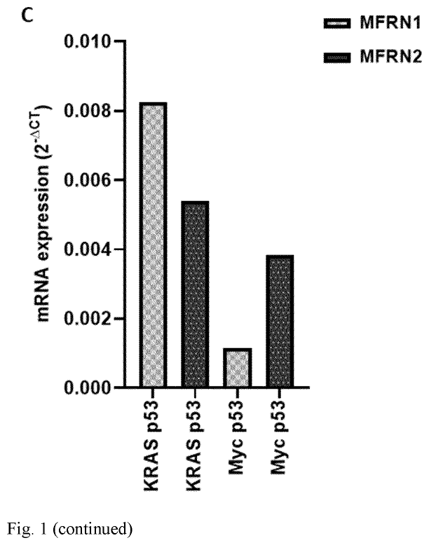

### Example 1, Relating to FIG. 1A

Protein lysates of the indicated cell lines were generated using cell lysis buffer (Cell Signaling Technology) supplemented with both protease (Complete Mini; Roche) and phosphatase inhibitors. To ensure lysis, cells were sonicated for 5 min on ice and subsequently centrifuged at 4° C. at 13,000 rpm to collect protein lysates. Furthermore, protein lysates were equalized utilizing BCA protein assay (Thermo Scientific), equal amount of protein were mixed with Laemmli buffer (100 mM Tris-HCl pH 6.8, 5% glycerol, 2% SDS, 5% 2-mercaptoethanol) and boiled at 95° C. for 5 min. Proteins were separated by SDS-PAGE, transferred onto PVDF membrane, and detected by immunoblotting using Anti-MFRNI antibody (Proteintech Catalogue number 26469-1-AP). Image detection was performed with AlphaView software (ProteinSimple) using the Clarity Western ECL substrate Solution (Bio-Rad).

### Example 2, Relating to FIGS. 1B and C

Total RNA was isolated using RNeasy Mini Kit (Qiagen) and Rnase-Free Dnase Set (Qiagen) in accordance to manufacturer's protocol. 1 μg of purified RNA was reverse transcribed using TaqMan® Reverse Transcription Reagents (Thermo Fisher Scientific) and diluted 1:20 before subjected to qPCR. For the qPCR reaction, cDNA was mixed with Power SYBR® Green Master Mix (Thermo Fisher Scientific) and target specific primers and performed in triplicate. Transcript levels were normalized to the levels of Tubulin mRNA expression and calculated using the deltaCt (ACt) method, qPCR was carried out using QuantStudio 3 Real-Time PCR system (Applied biosystems). Following primers were used

human-Mfrn1 ff qPCR
SEQ ID NO: 9
CGGTGGACTCGGTGAAGAC;
human-Mfrn1 rev qPCR
SEQ ID NO: 10
GGGCTCCGTAGATACTTGTGTA;
human-Mfrn2 ff qPCR
SEQ ID NO: 11
GGCTGAACGTCACAGCAAC;
human-Mfrn2 rev qPCR
SEQ ID NO: 12
GCACCATTGGCAATATGGCT;
mm-Mfrn1 ff qPCR
SEQ ID NO: 13
TTGAATCCAGATCCCAAAGC;
mm-Mfrn1 rev qPCR
SEQ ID NO: 14
GTTTCCTTGGTGGCTGAAAA;
mm-Mfrn2 ff qPCR
SEQ ID NO: 15
TCGTCAAGCAGAGGATGCAGAT;
mm-Mfrn2 rev qPCR
SEQ ID NO: 16
GTTAAAGTGCTCTTGCAGGAAC;

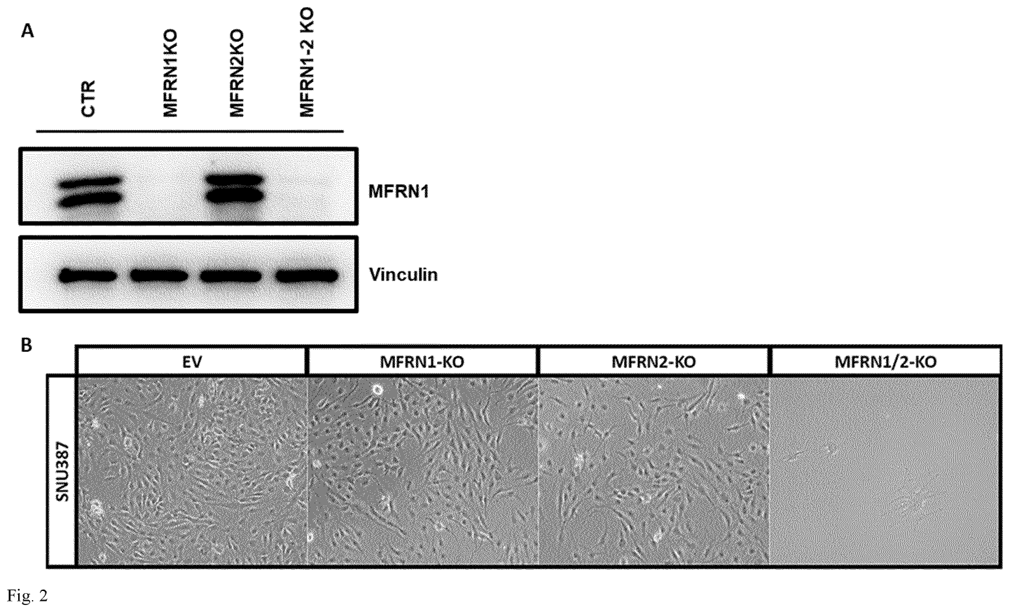

### Example 3, Relating to FIG. 2A

For lentivirus production, HEK293T cells were plated one day before transfection into 10 cm plates and transfected when near confluence was reached using a plasmid mix of 2.5 μg pMD.2G, 8 μg psPAX2 (Addgene plasmid #12259 and #12260) and 10 μg pLenti CRISPR v2 harboring respective guides in 1000 μl serum free DMEM and 60 μl polyethylenimine (PEI, 1 μg/μl). The SLC25A28 guide had the sequences 5′-caccGGTGACCGCCTATTTCCGAGtttAAGAGCTATGCTGGAAACAGCATAGCAAG TTTAAATAAGGCTAGTCCGTTATCAACTTGAAAAAGTGGCACCGAGTCGGTGCTT TTTT-3′ (SEQ ID NO:6).

The same results were obtained with a guide with the sequence 5′-caccGTTCAGGACGGTATATCAAGTGtttAAGAGCTATGCTGGAAACAGCATAGCA AGTTTAAATAAGGCTAGTCCGTTATCAACTTGAAAAAGTGGCACCGAGTCGGTGC TTTTTT-3′ (SEQ ID NO:8).

The plasmid mix was then vortexed for 5, incubated at room temperature for 30 min incubation and added drop-wise to cells. 24h following the transfection, medium was exchanged and lentiviral supernatant was harvested 48 h post-transfection using 0.45 μm Cellulose Acetate Membrane filters (VWR) and stored at −80° C. until use. SNU387 cells were plated on 10 cm plate and one day following the plating, cells were transduced with viral supernatants in the presence of Polybrene (4 μg/ml). 2 days post-transduction cells were selected with Puromycin (2 μg/ml). Three days after selection protein lysates were generated using cell lysis buffer (Cell Signaling Technology) supplemented with both protease (Complete Mini; Roche) and phosphatase inhibitors. To ensure lysis, cells were sonicated for 5 min on ice and subsequently centrifuged at 4° C. at 13,000 rpm to collect protein lysates. Furthermore, protein lysates were equalized utilizing BCA protein assay (Thermo Scientific), equal amount of protein were mixed with Laemmli buffer (100 mM Tris-HCl pH 6.8, 5% glycerol, 2% SDS, 5% 2-mercaptoethanol) and boiled at 95° C. for 5 min. Proteins were separated by SDS-PAGE, transferred onto PVDF membrane, and detected by immunoblotting using Anti-MFRNI antibody (Proteintech Catalogue number 26469-1-AP). Image detection was performed with AlphaView software (ProteinSimple) using the Clarity Western ECL substrate Solution (Bio-Rad).

### Example 4, Relating to FIG. 2B

Microscope images of SNU387 cells were obtained 8 days after CRISPR/Cas9 mediated KO of MFRN1 (MFRNIKO), MFRN2 (MFRN2KO) or both (MFRN1-2KO) (Example 3). EV=empty vector, was used as control.

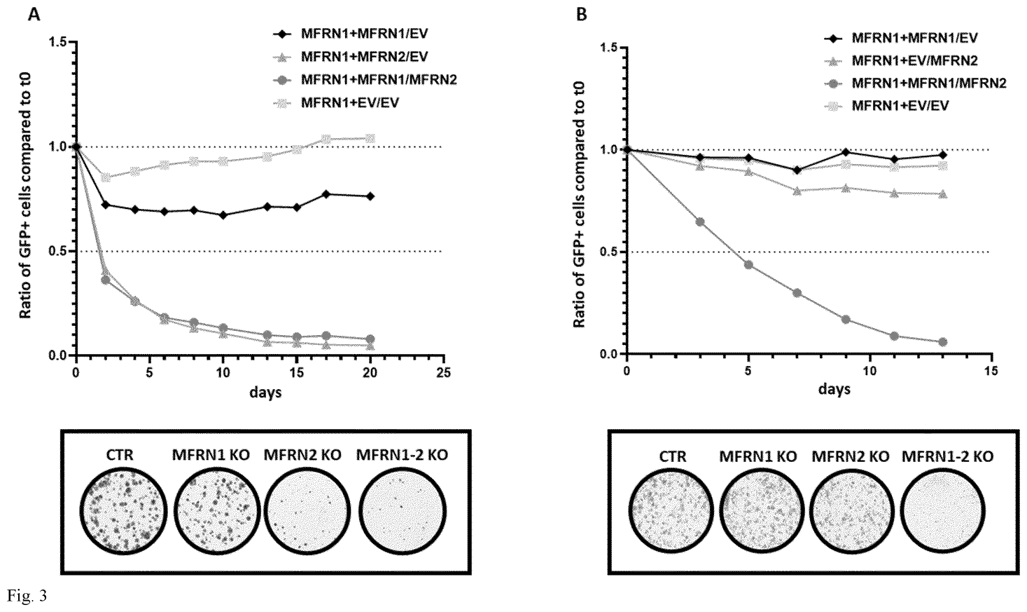

### Example 5, Relating to FIG. 3A

For lentivirus production, HEK293T cells were plated one day before transfection into 10 cm plates and transfected when near confluence was reached using a plasmid mix of 2.5 μg pMD.2G, 8 μg psPAX2 (Addgene plasmid #12259 and #12260) and 10 μg pLenti CRISPR v2 harboring respective guides in 1000 μl serum free DMEM and 60 μl polyethylenimine (PEI, 1 μg/μl), the SLC25A28 guides were the same as in Example 3. The plasmid mix was then vortexed for 5, incubated at room temperature for 30 min incubation and added drop-wise to cells. 24h following the transfection, medium was exchanged and lentiviral supernatant was harvested 48 h post-transfection using 0.45 μm Cellulose Acetate Membrane filters (VWR) and stored at −80° C. until use. HUH6 cells were plated on 10 cm plate and one day following the plating, cells were transduced with viral supernatants in the presence of Polybrene (4 μg/ml). 2 days post-transduction cells were selected with Puromycin (2 μg/ml). Three days after selection MFRNIKO cells were mixed with either MFRNIKO (MFRN1+MFRN1/EV), MFRN2KO (MFRN1+MFRN2/EV) or MFRN1 and 2 KO (MFRN1+MFRN1/MFRN2) cells each co-expressing GFP as a marker and the relative distribution of GFP expressing cells over time was measured using a Guava® easyCyte benchtop flow cytometer (Merck Millipore). To assess colony formation capacity, 500 cells were plated in 6 well plates as triplicate. Cells were fixed with methanol and stained with 0.05% crystal violet after 10 days.

### Example 6, Relating to FIG. 3B

For lentivirus production, proceeding was essential as in Example 5. SNU387 cells were plated on 10 cm plate and one day following the plating, cells were transduced with viral supernatants in the presence of Polybrene (4 μg/ml). 2 days post-transduction cells were selected with Puromycin (2 μg/ml). Three days after selection MFRNIKO cells were mixed with either MFRNIKO (MFRN1+MFRN1/EV), MFRN2KO (MFRN1+MFRN2/EV) or MFRN1 and 2 KO (MFRN1+MFRN1/MFRN2) cells each co-expressing GFP as a marker and the relative distribution of GFP expressing cells over time was measured using a Guava® easyCyte benchtop flow cytometer (Merck Millipore). To assess colony formation capacity, 500 cells were plated in 6 well plates as triplicate. Cells were fixed with methanol and stained with 0.05% crystal violet after 10 days.

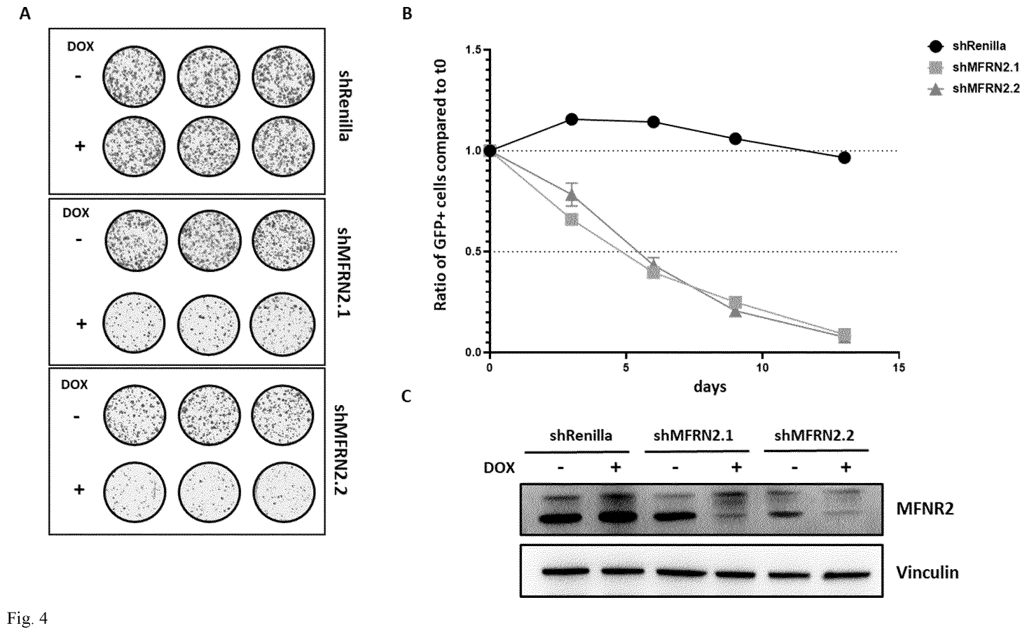

### Example 7, Relating to FIG. 4A

For Retrovirus production, HEK-gp-cells were one day before transfection into 10 cm plates and transfected when near confluence was reached using a plasmid mix of 2.5 μg pMD.2G and 20 μg retroviral plasmid LT3GEPIR with respective shRNAs in 1000 μl serum free DMEM and 60 μl polyethylenimine (PEI, 1 μg/μl). The SLC25A28 shRNA expression constructs had the sequences

5′-ctcgactagggataacagggtaattgtttgaatgaggcttcagtactttacagaatcgttgcctgcacatcttggaaacacttgctggg attacttcgacttcttaacccaacagaaggctcgagaaggtatattgctgttgacagtgagcgcTGCTGTTGACAGTGAG CGCGGCAAGTGAAGTAGCACTGAATAGTGAAGCCACAGATGTATTCAGTGCTACT TCACTTGCCATGCCTACTGCCTCGGAatgcctactgcctcggacttcaaggggctagaattcgagcaattatctt gtttactaaaactgaataccttgctatctctttgatacatttttacaaagctgaattaaaatggtataaattaaatcactttttt-3′ (SEQ ID NO:2, shMFRN2.1) and
5′-ctcgactagggataacagggtaattgtttgaatgaggcttcagtactttacagaatcgttgcctgcacatcttggaaacacttgctggg attacttcgacttcttaacccaacagaaggctcgagaaggtatattgctgttgacagtgagcgcTGCTGTTGACAGTGAG CGCTCCTAATAAAAAGCCTTTAAATAGTGAAGCCACAGATGTATTTAAAGGCTTT TTATTAGGAATGCCTACTGCCTCGGAatgcctactgcctcggacttcaaggggctagaattcgagcaattatctt gtttactaaaactgaataccttgctatctctttgatacatttttacaaagctgaattaaaatggtataaattaaatcactttttt-3′ (SEQ ID NO:4, shMFRN2.2).

The plasmid mix was then vortexed for 5, incubated at room temperature for 30 min incubation and added drop-wise to cells. 24h following the transfection, medium was exchanged and lentiviral supernatant was harvested 48 h post-transfection using 0.45 μm Cellulose Acetate Membrane filters (VWR) and stored at −80° C. until use. HUH6 cells were plated on 10 cm plate and one day following the plating, cells were transduced with viral supernatants in the presence of Polybrene (4 μg/ml). 2 days post-transduction cells were selected with Puromycin (2 μg/ml). Three days after selection 500 cells were plated in 6 well plates as triplicate and cultured with or without doxycycline supplementation to allow shRNA expression (1 μg/ml). Cells were fixed with methanol and stained with 0.05% crystal violet after 10 days.

### Example 7, Relating to FIG. 4B

HUH6 cells transduced with the indicated shRNAs were mixed with parental, non-transduced cells and cultured in the presence of doxycycline (1 μg/ml) to allow shRNA and GFP expression in transduced cells. The relative distribution of GFP expressing cells over time was measured using a Guava® easyCyte benchtop flow cytometer (Merck Millipore) over time.

### Example 8, Relating to FIG. 4C

HuH6 cells with indicated shRNA constructs were grown 4 days with or without DOX and protein lysates were generated using cell lysis buffer (Cell Signaling Technology) supplemented with both protease (Complete Mini; Roche) and phosphatase inhibitors. To ensure lysis, cells were sonicated for 5 min in ice and subsequently centrifuged at 4° C. at 13,000 rpm to collect protein lysates. Furthermore, protein lysates were equalized utilizing BCA protein assay (Thermo Scientific), equal amount of protein were mixed with Laemmli buffer (100 mM Tris-HCl pH 6.8, 5% glycerol, 2% SDS, 5% 2-mercaptoethanol) and boiled at 95° C. for 5 min. Proteins were separated by SDS-PAGE, transferred onto PVDF membrane, and detected by immunoblotting using Anti-MFRN2 antibody (Abcam Catalogue number ab80467). Image detection was performed with Alpha View software (ProteinSimple) using the Clarity Western ECL substrate Solution (Bio-Rad).

### Example 9: In Vivo Experiments

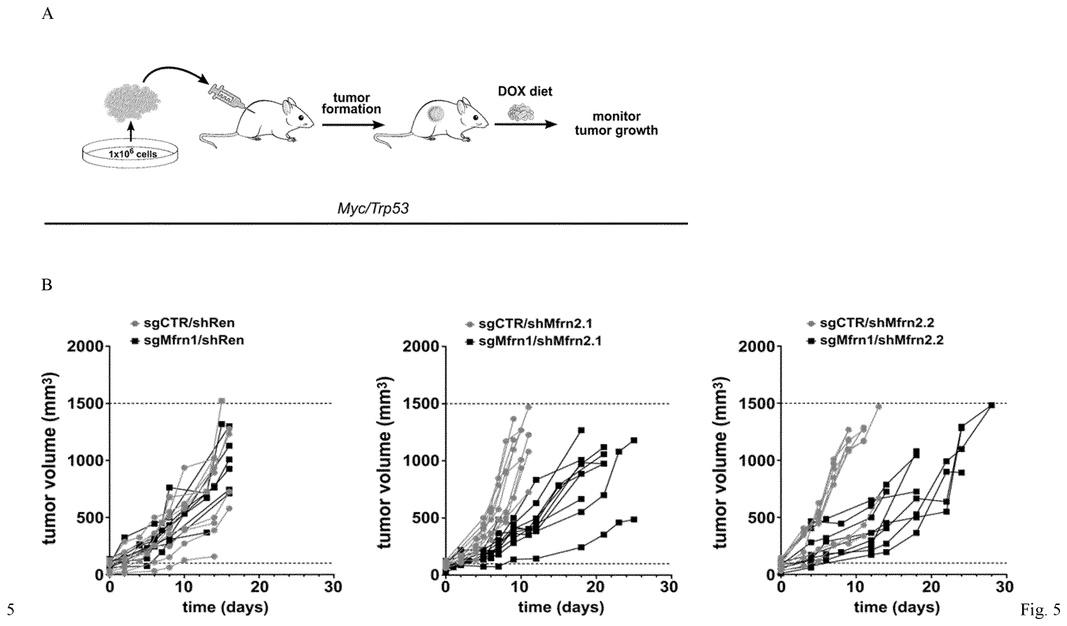

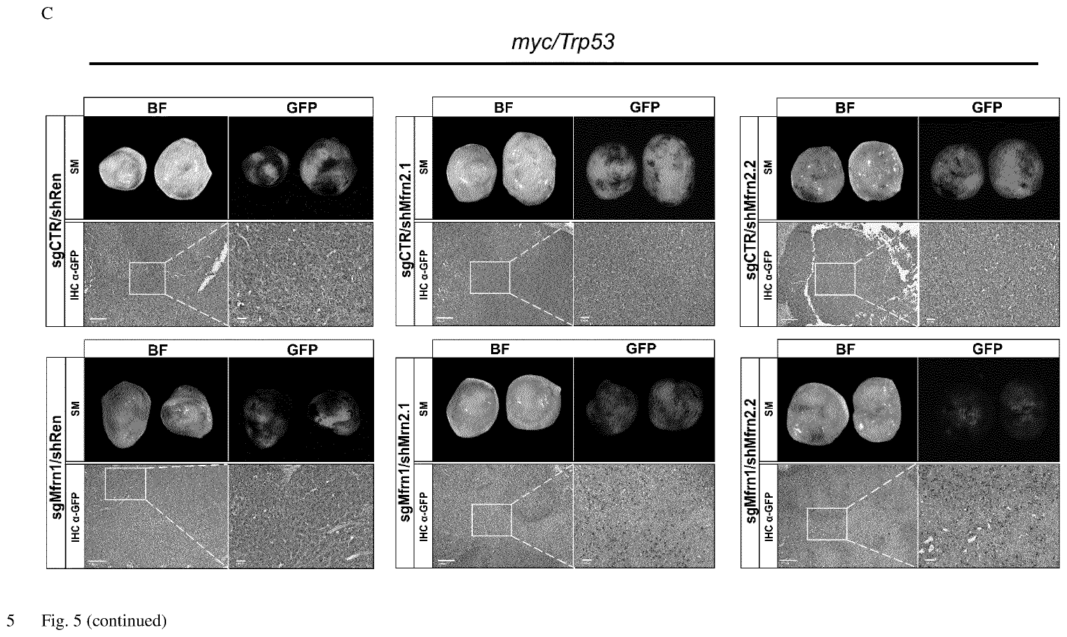

In relation to FIG. 5, primary murine Myc/Trp53 liver cancer cells stably expressing SpCas9 with either sgMfrn1 or sgCTR together with a construct expression either shMfrn2.1, shMfrn2.2 or shRenilla (shRen) were sc, injected into NMRI nude mice (n=5 female mice per group). After reaching a tumor volume of 100 mm3 mice were given a DOX containing diet and tumor size was measured periodically. As shown in FIG. 5(B), xenografts with a double MFRNI and MFRN2 depletion show a marked growth impediment.

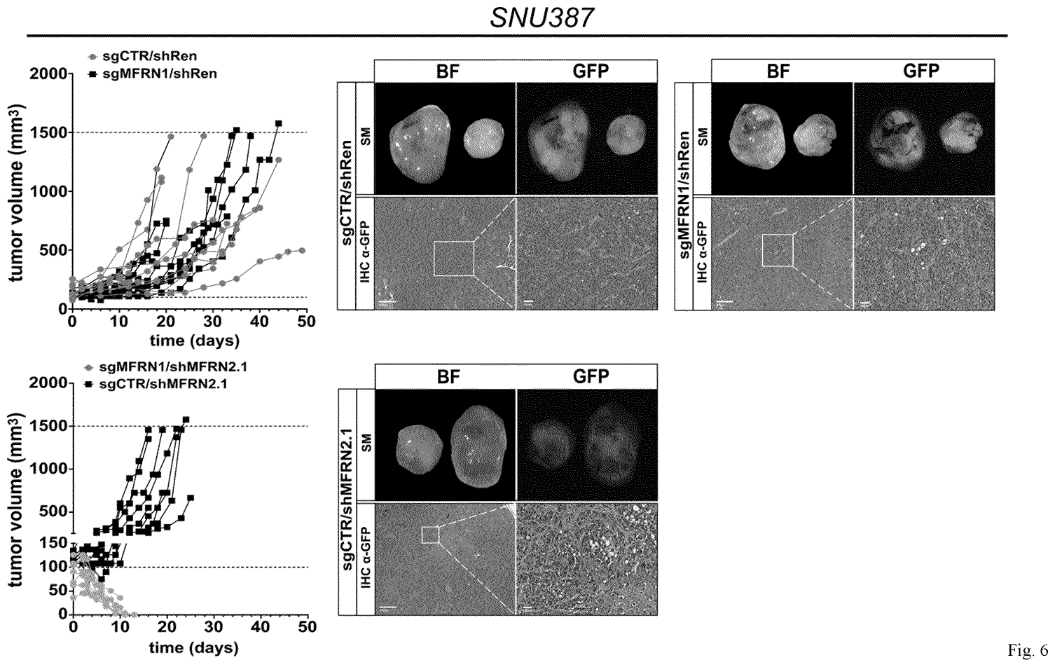

In relation to FIG. 6, SNU387 cells stably expressing SpCas9 with either sgMFRNI or sgCTR together with a construct expression either shMFRN2 or shRenilla (shRen) were sc, injected into NSG nude mice (n=5 female mice per group). After reaching a tumor volume of 100 mm3 mice were given a DOX containing diet and tumor size measured periodically. As shown in FIG. 6, xenografts with a double MFRNI and MFRN2 depletion show complete regression in almost all cases.

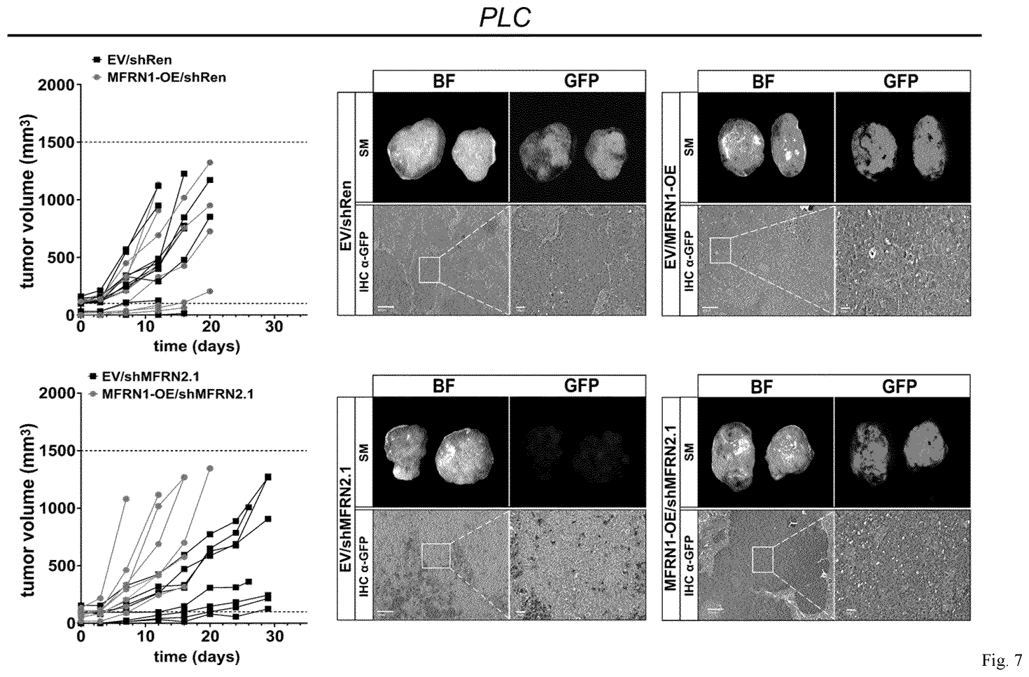

IN relation to FIG. 7, PLC cells stably expressing MFRNI cDNA or the empty expression cassette (EV) together with a construct expression either shMFRN2 or shRen were sc, injected into NSG nude mice (n=5 female mice per group). After reaching a tumor volume of 100 mm3 mice were given a DOX containing diet and tumor size measured periodically. As shown in FIG. 7, MFRNI overexpression can rescue the growth impairment induced by MFRN2 depletion.

### Example 10: MFRNI Expression in Tumor Samples

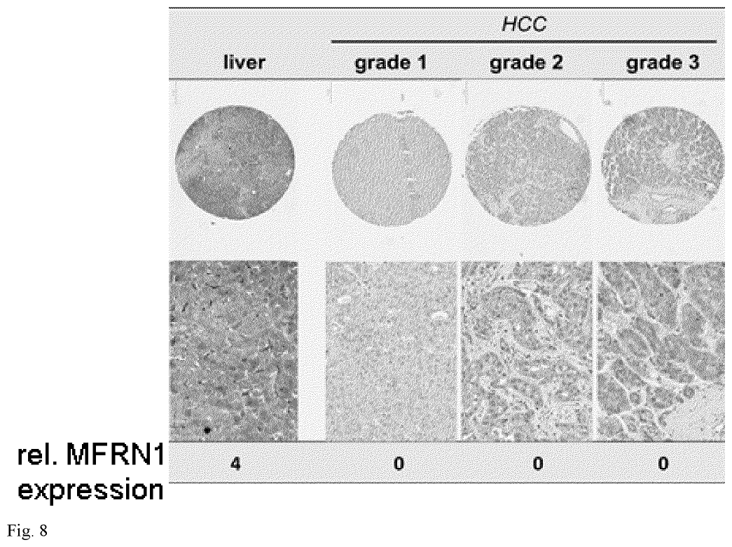

Human samples from normal liver tissue and various grades of hepatocellular carcinoma (HCC) were stained for MFRNI expression and allocated to one of five groups of MFRNI expression levels (O: not detectable, 1: low, 2:medium, 3: high, and 4 very high expression). As shown in FIG. 8, normal liver tissue on average has high MFRN1 exprssion, while HCC samples show significantly lower espression. E.g. in grade 3 HCC samples, more than 70% of the samples were found to have undetectable or low MFRN1 expression.

### LITERATURE CITED

Elbashir et al. (2001), Nature 411: 494-498
Hammond et al. (2001), Nature Rev. Genet. 2, 110-119
Kang et al. (2019), Autophagy 15(1): 172
Klevenz et al., Cell Mol Life Sci. 2002, 59: 1993-1998
Li et al. (2018), Dev Cell 46(4):441
Shen et al. (2018), J Cell Biochem 119(11):9178

1. A method for treating a cancer with reduced activity of mitoferrin-1 in a subject comprising administering to the subject an inhibitor of mitoferrin-2.

2. The method of claim 1, wherein said subject is a human.

3. The method of claim 2, wherein said cancer with reduced activity of mitoferrin-1 is a cancer comprising a deletion of chromosome 8p.

4. The method of claim 1, wherein said cancer is a solid cancer, preferably is liver cancer, lung cancer, pancreas cancer, or colon cancer.

5. The method of claim 1, wherein said inhibitor of mitoferrin-2 comprises a polynucleotide, a polypeptide, and/or an iron chelator.

6. The method of claim 1, wherein said polynucleotide has the activity of reducing expression of the gene encoding mitoferrin-2.

7. The method of claim 1, wherein said inhibitor of mitoferrin-2 mediates at least one of (i) an at least partial knock-out of the gene encoding mitoferrin-2, (ii) RNA interference of mitoferrin-2 gene expression, and (iii) silencing of mitoferrin-2 gene expression.

8. The method of claim 1, wherein said inhibitor of mitoferrin-2 comprises an siRNA, a shRNA, and/or an miRNA.

9. The method of claim 1, wherein said inhibitor of mitoferrin-2 comprises an aptamer, an antibody, or a fragment thereof.

10. The method of claim 1, wherein said inhibitor of mitoferrin-2 is a specific inhibitor of mitoferrin-2.

11. A method for identifying an inhibitor of mitoferrin-2 comprising
(a) contacting a (i) host cell with a reduced mitoferrin-1 activity and (ii) a host cell with a non-reduced mitoferrin-1 activity with a candidate inhibitor of mitoferrin-2,
(b) determining growth and/or morphology of the host cells of step (a); and
(c) identifying an inhibitor of mitoferrin-2 if a growth arrest and/or abnormal morphology is/are detected in step (b) in the host cell having the reduced activity of mitoferrin-1 but not in the host cell with the non-reduced activity of mitoferrin-1.

12. The method of claim 11, wherein said cell with a reduced mitoferrin-1 activity is a human cell comprising a chromosome 8p deletion.

13. A method for identifying a subject susceptible to cancer treatment by an inhibitor of mitoferrin-2, comprising
(A) determining mitoferrin-1 activity in a sample of said subject, preferably a sample of cancer cells, and
(B) identifying a patient susceptible to cancer treatment by an inhibitor of mitoferrin-2 based on determining step (A).

14. The method of claim 13, wherein said reference is a sample of cells known to be susceptible to treatment by an inhibitor of mitoferrin-2, and wherein a patient susceptible to cancer treatment by an inhibitor of mitoferrin-2 is identified if the expression determined in step (A) is essentially identical to or lower than the reference; or wherein said reference is a sample of cells known not to be susceptible to treatment by an inhibitor of mitoferrin-2, and wherein a patient susceptible to cancer treatment by an inhibitor of mitoferrin-2 is identified if the expression determined in step (A) is lower than the reference.

15. (canceled)


SEQUENCE LISTING (16 sequences)

SEQ ID NO: 1 - DNA, 22 bp, Artificial Sequence
  SLC25A28 shRNA1
ttcagtgctacttcacttgcca

SEQ ID NO: 2 - DNA, 384 bp, Artificial Sequence
  SLC25A28 shRNA1 long
ctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgctgctgttgacagtgagcgcggcaagtgaagtagcactgaatagtgaagccacagatgtattcagtgctacttcacttgccatgcctactgcctcggaatgcctactgcctcggacttcaaggggctagaattcgagcaattatcttgtttactaaaactgaataccttgctatctctttgatacatttttacaaagctgaattaaaatggtataaattaaatcactttttt

SEQ ID NO: 3 - DNA, 22 bp, Artificial Sequence
  SLC25A28 shRNA2
tttaaaggctttttattaggaa

SEQ ID NO: 4 - DNA, 384 bp, Artificial Sequence
  SLC25A28 shRNA2 long
ctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgctgctgttgacagtgagcgctcctaataaaaagcctttaaatagtgaagccacagatgtatttaaaggctttttattaggaatgcctactgcctcggaatgcctactgcctcggacttcaaggggctagaattcgagcaattatcttgtttactaaaactgaataccttgctatctctttgatacatttttacaaagctgaattaaaatggtataaattaaatcactttttt

SEQ ID NO: 5 - DNA, 20 bp, Artificial Sequence
  Slc25a28 G1
ggtgaccgcctatttccgag

SEQ ID NO: 6 - DNA, 115 bp, Artificial Sequence
  Slc28a28 G1 long
caccggtgaccgcctatttccgagtttaagagctatgctggaaacagcatagcaagtttaaataaggctagtccgttatcaacttgaaaaagtggcaccgagtcggtgctttttt

SEQ ID NO: 7 - DNA, 20 bp, Artificial Sequence
  Slc25a28 G2
ttcaggacggtatatcaagt

SEQ ID NO: 8 - DNA, 117 bp, Artificial Sequence
  Slc28a28 G2 long
caccgttcaggacggtatatcaagtgtttaagagctatgctggaaacagcatagcaagtttaaataaggctagtccgttatcaacttgaaaaagtggcaccgagtcggtgctttttt

SEQ ID NO: 9 - DNA, 19 bp, Artificial Sequence
  human-Mfrn1 ff
cggtggactcggtgaagac

SEQ ID NO: 10 - DNA, 22 bp, Artificial Sequence
  human-Mfrn1 rev
gggctccgtagatacttgtgta

SEQ ID NO: 11 - DNA, 19 bp, Artificial Sequence
  human-Mfrn2 ff
ggctgaacgtcacagcaac

SEQ ID NO: 12 - DNA, 20 bp, Artificial Sequence
  human-Mfrn2 rev
gcaccattggcaatatggct

SEQ ID NO: 13 - DNA, 20 bp, Artificial Sequence
  mm-Mfrn1 ff
ttgaatccagatcccaaagc

SEQ ID NO: 14 - DNA, 20 bp, Artificial Sequence
  mm-Mfrn1
gtttccttggtggctgaaaa

SEQ ID NO: 15 - DNA, 22 bp, Artificial Sequence
  mm-Mfrn2
tcgtcaagcagaggatgcagat

SEQ ID NO: 16 - DNA, 22 bp, Artificial Sequence
  mm-Mfrn2 rev
gttaaagtgctcttgcaggaac